(19) United States
(12) Patent Application Publication
(10) Pub. No.: US 2005/0137575 A1
(43) Pub. Date: Jun. 23, 2005

(54) Minimally invasive injection devices and methods

(75) Inventors: Richard B. Thompson, Durham, NC (US); Doris A. Taylor, Durham, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Durham, NC (US)

(21) Appl. No.: 10/738,212

(22) Filed: Dec. 18, 2003

(51) Int. Cl. A61M 31/00

(52) U.S. Cl. 604/522; 604/187; 600/427; 600/114

(57) ABSTRACT

Devices and methods whereby therapeutic agents may be injected into distinct organ locations in a minimally invasive manner. Most preferably the devices and methods employ a video-assisted thorascopic system (VATS) to enable a physician in real time to visually identify a distinct organ location into which therapeutic agent is to be injected. In particularly preferred forms, devices are provided for injecting a therapeutic agent into a tissue site which include a proximal handle, and a tubular barrel distally extending from the handle. The barrel has an injection needle at a distal end thereof which is most preferably angled relative to the barrel's elongate axis. The internal space of the barrel is sized and configured to receive a cartridge containing a therapeutic agent to be injected into the tissue site. A plunger assembly and injection trigger assembly are provided so as to cause the plunger to expel a predetermined volume of the therapeutic agent from the cartridge to the needle and thereby allow injection thereof to the tissue site in response to operation of the trigger assembly.

### FIELD OF THE INVENTION

The present invention relates generally to the field of medical devices and methods. In especially preferred forms, the present invention relates to minimally invasive devices and methods to permit localized injections of a therapeutic agent.

### BACKGROUND AND SUMMARY OF THE INVENTION

Cell transplantation to repopulate injured myocardium has shown to be an effective therapy for improving both systolic and diastolic ventricular function pre-clinically. Current cell delivery methods are either via an open surgical approach during coronary artery bypass or left ventricular assist device insertion or via a variation of endoventricular catheter delivery. Each of these conventional methods has benefits and limitations. For example, percutaneous catheter delivery of cells has the benefit of being minimally invasive but lacks direct visualization of the cell injection region. Open surgical delivery necessitates a sternotomy or thoracotomy but allows precise delivery of cells to the myocardium.

It would therefore be highly desirable if devices and/or methods could be provided which would allow the real time visual delivery of cells (or other therapeutic agents) to distinct organ locations in a minimally invasive manner. Such devices and/or methods could thereby obtain the benefits of precise therapeutic agent delivery under direct visualization. It is towards fulfilling such needs that the present invention is directed.

Broadly, the present invention is embodied in devices and methods whereby therapeutic agents may be injected into distinct organ locations in a minimally invasive manner. Most preferably the devices and methods of this invention employ a video-assisted thorascopic system (VATS) to enable a physician in real time to visually identify a distinct organ location into which therapeutic agent is to be injected.

In particularly preferred forms, the present invention is embodied in devices for injecting a therapeutic agent into a tissue site which include a proximal handle, and a tubular barrel distally extending from the handle. The barrel has an injection needle at a distal end thereof which is most preferably angled relative to the barrel's elongate axis. The internal space of the barrel is sized and configured to receive a cartridge containing a therapeutic agent to be injected into the tissue site. A plunger assembly and injection trigger assembly are provided so as to cause the plunger to expel a predetermined volume of the therapeutic agent from the cartridge to the needle and thereby allow injection thereof to the tissue site in response to operation of the trigger assembly.

A position dial may be provided so as to operably interconnect the barrel to the handle and to allow the barrel to be rotated about its longitudinal axis. Rotation of the position dial will therefore cause the barrel to rotate thereby allowing a selective change in the relative angular orientations of the injection needle.

In order to prevent inadvertent needle punctures while the device is being maneuvered, a needle guard is most preferably provided so as to sheath the needle. The needle guard is thus moveable between an advanced position wherein the injection needle is sheathed thereby, and a retracted position wherein the injection needle is exposed. Movements of the needle guard may be achieved by means of a pivotally moveable actuator lever attached to the patient external handle. An actuator rod operatively interconnects the actuator lever and the needle guard. As such, pivotal movements of actuator lever responsively moves the needle guard between its advanced and retracted positions.

These and other aspects and advantages will become more apparent after careful consideration is given to the following detailed description of the preferred exemplary embodiments thereof.

### BRIEF DESCRIPTION OF THE ACCOMPANYING DRAWINGS

Reference will hereinafter be made to the accompanying drawings, wherein like reference numerals throughout the various FIGURES denote like structural elements, and wherein;

FIG. 1 is a side elevational view of an injection device in accordance with the present invention;

FIG. 2 is a view of the internal mechanisms associated with the device shown in FIG. 1;

FIG. 3 is a greatly enlarged schematic view of a technique in accordance with the present invention for injecting a therapeutic agent into a discrete organ location.

### DETAILED DESCRIPTION OF THE INVENTION

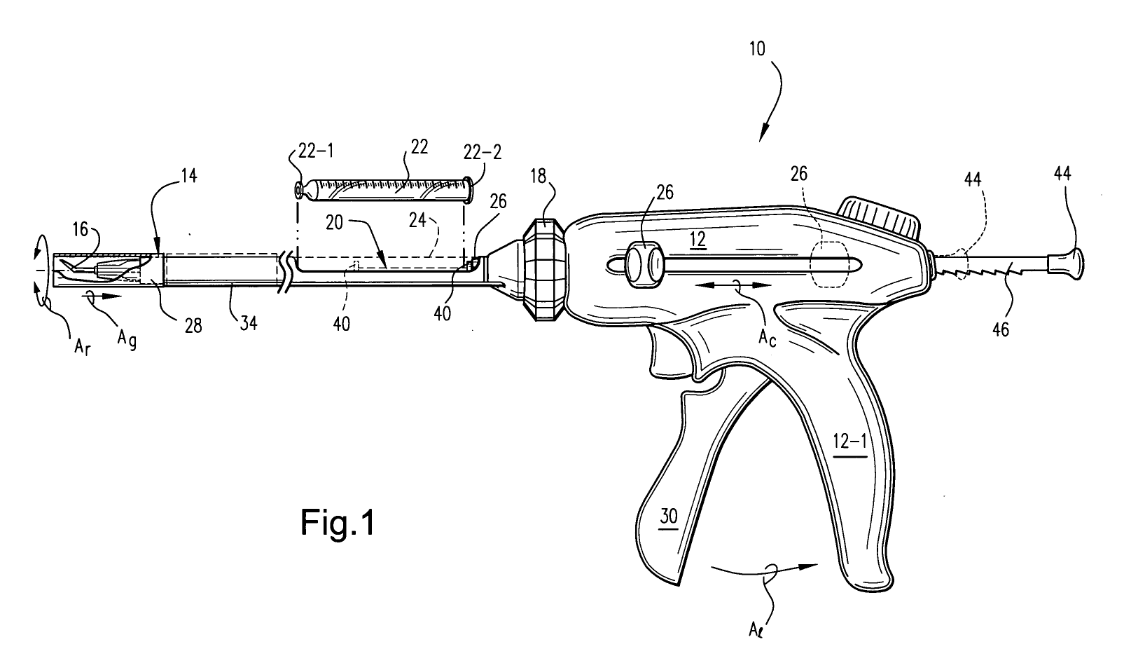

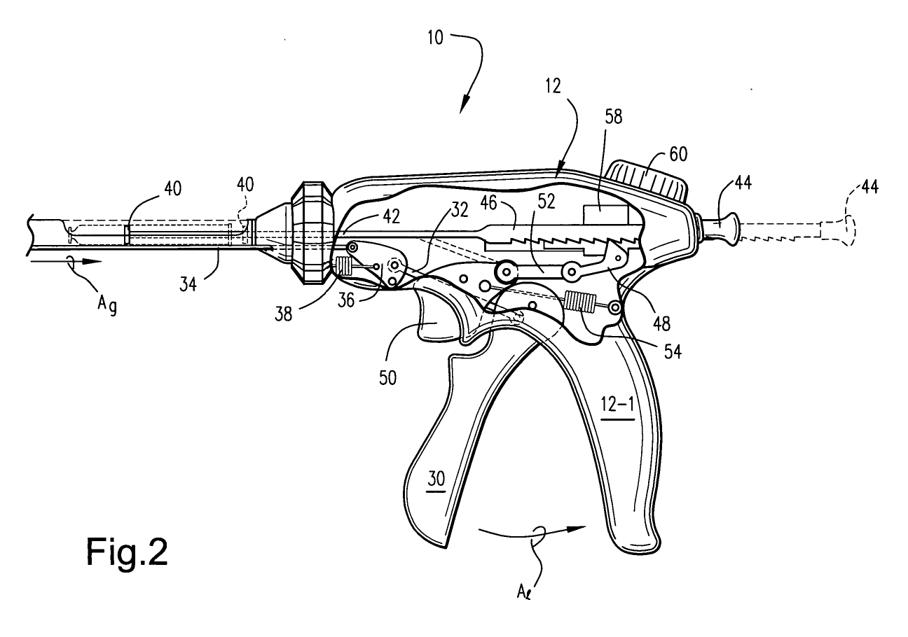

One particularly preferred form of an injection device 10 is depicted in accompanying FIGS. 1 and 2. Specifically, the device 10 includes a proximal handle body 12 which includes a depending pistol grip 12-1 sized and configured to be manually grasped and manipulated by an attending physician. An elongate rigid tubular barrel 14 extends distally from the handle 12 and terminates in a distally located angled injection needle 16. The length of the barrel 14 is of course sufficient to allow placement of the distal injection needle 16 in the organ of interest. For example, when configured to inject cells into myocardium, the barrel length may be about 15 cm ± in length.

The handle 12 and barrel 14 are joined to one another via a rotatable dial 18. More specifically, the rotatable dial 18 is coaxially fixed to the barrel 14 and is rotatable with respect to the handle 12. Thus, the barrel 14 and dial 18 may be rotated about the barrel's elongate axis (arrow Aᵣ) as a unit so as to allow the physician to change the orientation of the angled injection needle 16 as may be desired to facilitate its placement and injection of the therapeutic agent. Most preferably, the dial 18 may be provided with position markings to assist the physician in determining the relative positioning of the distal needle 16.

A cartridge loading chamber 20 is provided in proximal axial section of the barrel 14. The loading chamber 20 thus allows a cartridge or vial 22 containing the therapeutic agent (e.g., cells) to be loaded into the hollow of the barrel 14 proximally of the needle 16. When positioned within the barrel 14, the discharge port 22-1 of the cartridge 22 will therefore be in communication with the injection needle 16 to allow the contents of the cartridge 22 to be transferred to and through the needle 16. A cover 24 attached to knob 26 allows the loading chamber 20 to be reciprocally slideably moved (arrow A_{c}) between an opened condition (as shown in solid line in FIG. 1) and a closed condition (as shown in dashed line in FIG. 1). While in the opened condition, therefore, the cartridge 22 may be positioned within the loading chamber, such that upon movement of the handle 26 the cover 24 will close the chamber 20 thereby retaining the cartridge 22 therein.

A tubular needle guard 28 is sleeved over a distal section of the barrel 14. As is perhaps better shown in FIG. 2, the needle guard 28 is connected to an actuator lever 30 pivotally connected to the handle 12 by means of proximal and distal actuator rods 32, 34 and a pivotal linkage 36. A tension spring 38 is connected operatively to the linkage 36 so as to bias its pivotal movement in a direction tending to cause the guard 28 to be in its normal advanced position with respect to needle 16.

As shown in solid line in FIG. 1, the needle guard 28 is normally in an advanced position whereby the needle 16 is covered. However, manually squeezing the lever 30 will cause it to be moved pivotally in the direction of arrow Aₗ (see FIGS. 1 and 2) toward the grip 12-1. Such movement of the actuator 30 will responsively slideably move the guard 28 in a proximal direction (arrow A_{g} in FIGS. 1 and 2) by virtue of the responsive retraction of the actuator rods 32, 34 and clockwise pivoting (as viewed in FIG. 2) of the linkage 36 thereby exposing the needle 16. In such a manner, the physician may selectively cover the needle 16 with the guard 28 so as to maneuver the needle 16 from one organ injection site to another safely and yet expose the needle once a new injection site has been located. The tension spring 38 ensure that, once manual squeezing pressure is released from the lever 30, the lever 30 and hence the needle guard 28 will return to their respective normal conditions as shown in solid line in FIGS. 1 and 2.

A plunger 40 is provided at the distal-most end of a plunger control rod 42 mounted within the handle 12 for reciprocal rectilinear movements. The proximal-most end of the control rod 42 carries a knob 44. The control rod 42 also includes a longitudinal section of ratchet teeth 46 engageable with ratchet pawl 48. The knob 44 allows the control rod 42, and hence the distal plunger 40 thereof, to be pulled manually into a retracted position as shown in solid line in FIG. 1, for example, by twisting the control rod 42 one quarter-turn so as to disengage the ratchet teeth and pawl 46, 48, respectively, and then pulling rearwardly. Once in the retracted position, the quarter-turn may be reversed to thereby again re-engage the ratchet teeth and pawl 46, 48, respectively.

Controlled advancement of the control rod 42, and hence the distal plunger 40 thereof, is achieved by operation of the engaged ratchet teeth 46 and pawl 48 by means of the injection trigger 50. As can be seen more clearly in FIG. 2, the pawl 48 and trigger 50 are each pivotally attached to the handle 12 at one location, and are pivotally interconnected to one another at another location via pivot linkage 52. A tension spring 54 is connected operatively to the injection trigger 50 so as to bias it into its inactive position (shown in solid line in FIG. 2). Pulling on the trigger 50 against the bias force of the spring 54 will therefore cause the pawl 48 to be pivotally moved via linkage 52 out of engagement with one of the ratchet teeth 46. Releasing the trigger 50 will allow the bias force of the spring 54 to return the trigger to its inactive position which in turn responsively causes the pawl 48 to pivot back into engagement with a distally successive one of the ratchet teeth 46 and thereby forwardly advance the control rod an incremental longitudinal distance. As a result, the plunger will cause a corresponding incremental volume of therapeutic agent to be forcible expelled through the needle 16.

The forward distance which the plunger advanced each time the trigger 50 is depressed and released is determined by the geometries of the teeth 46 and pawl 48, as well as the dimensional “throw” of the pawl 48 (e.g., the number of adjacent teeth that are by-passed by the pawl when it disengages from a tooth and then re-engages with another one of the teeth proximally thereto). Most preferably, the pawl 48 is pivotally connected to a longitudinally slideable control member 58 which, in turn, is connected operatively to volume control dial 60. Thus, turning movement applied to the control dial 60 will translate into longitudinal movements distally or proximally (i.e., in dependence upon the direction of turning movement applied to the dial 60) of the control member 58. This longitudinal movement of control member 58 will thereby either increase or decrease the effective “throw” of the pawl 48 each time the trigger 50 is operated. Thus, by turning the control dial 60, the pawl 48 may be caused to engage either the immediately next successive one of the teeth 46, or every second, third, fourth etc. one of the teeth 46 each time the trigger 50 is operated. In such a manner, therefore, an attending physician may preselect the volume of therapeutic agent which is expelled from the needle 16 for each operable cycle of the trigger 50.

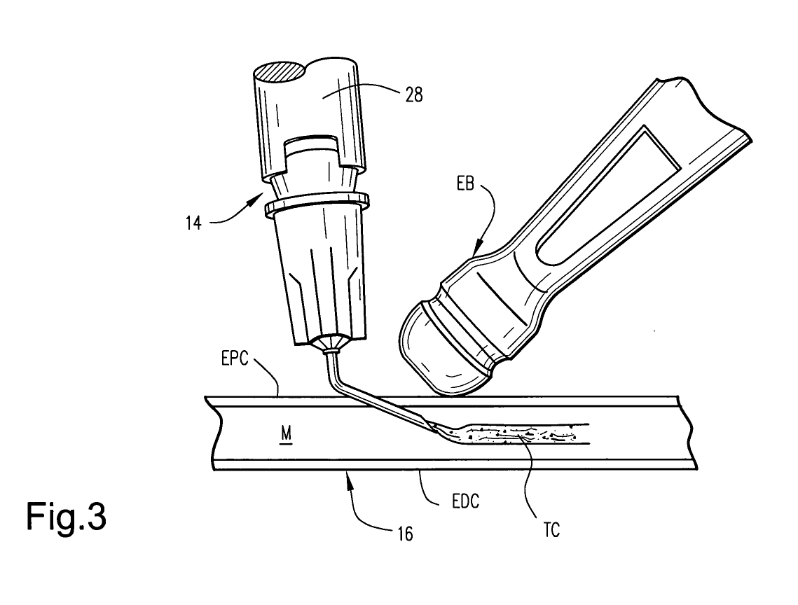

In use, the distal end of the barrel is inserted through a minimally invasive surgical incision near the organ to be injected. Thus, when injecting cells into myocardium, minimally invasive cardiac procedures may be employed which are well known to those in this art. The attending physician guides the distal end of the barrel 14 with the needle 16 sheathed by guard 28 under thorascopic visual observation using a patient external monitor and patient internal video probe (not shown) associated with a video-assisted thorascopic system (VATS). Once the site for injection has been determined the needle 16 may be unsheathed by retraction of the guard 28 and the relative angular orientation of the needle 16 changed as may be desired or necessary by manipulation of the dial 18. As shown in FIG. 3, the needle 16 may then be advanced into the myocardium M between the epicardium EPC and endocardium EDC tissue layers. The physician will select the volume amount of transplanted cells to be injected by manipulation of the volume control dial 60 and then operate the trigger 50 thereby causing such desired amount of transplanted cells TC to be injected into the myocardium M. The myocardium M may be stabilized physically by placement of an endo babcock EB adjacent the injection site.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A device for injecting a therapeutic agent into a tissue site, comprising:
a proximal handle;
a tubular barrel distally extending from the handle and having an injection needle at a distal end thereof, said barrel being sized and configured to receive a cartridge containing a therapeutic agent to be injected into the tissue site;
a plunger assembly that is moveable into an operable condition relative to the cartridge; and
an injection trigger assembly connected to said plunger and operable to responsively cause the plunger to expel a predetermined volume of the therapeutic agent from the cartridge to the needle and thereby allow injection thereof to the tissue site.

2. The device of claim 1, wherein said injection needle is angled with respect to a longitudinal axis of the barrel.

3. The device of claim 2, further comprising a position dial which operably connects the barrel to the handle and which allows the barrel to be rotated about said longitudinal axis thereof so as to change relative angular orientations of the injection needle.

4. The device of claim 1, further comprising a needle guard moveable between an advanced position wherein the injection needle is sheathed thereby, and a retracted position wherein the injection needle is exposed.

5. The device of claim 4, wherein said handle includes a pivotally moveable actuator lever, and an actuator rod operatively interconnecting said actuator lever and said needle guard, wherein pivotal movements of said actuator lever responsively moves said needle guard between said advanced and retracted positions.

6. The device of claim 1, wherein said barrel includes a cartridge loading chamber to allow the cartridge containing the therapeutic agent to be injected to be loaded into the barrel.

7. The device of claim 6, wherein said barrel includes a chamber cover reciprocally slideably moveable between an opened and closed conditions to thereby allow and prevent, respectively, access to the cartridge loading chamber.

8. The device of claim 7, wherein said handle includes a reciprocally moveable knob connected operatively to said chamber cover so as to move the chamber cover between said opened and closed conditions in response to manual reciprocal movements of said knob.

9. The device of claim 1, wherein said plunger assembly includes a plunger control rod having a plunger at a distal end thereof and a plunger knob at a proximal end thereof externally of said handle.

10. The device of claim 9, wherein injection trigger assembly includes a trigger pivotally moveable between inactive and active positions, and a pawl connected operably to said trigger, and wherein said plunger control rod includes a longitudinal section of ratchet teeth, wherein said pawl is pivotally operable into and out of engagement with said ratchet teeth in response to movements of said trigger between said active and inactive positions thereof so as to longitudinally advance said plunger control rod a predetermined distance.

11. The device of claim 10, further comprising a volume control assembly which includes a slideable control member connected to the pawl, and a volume control dial operably connected to the control member, wherein turning movements applied to volume control dial moves the control member longitudinally thereby changing the orientation of the pawl relative to the ratchet teeth and thereby establishing said predetermined distance that the plunger control rod advances when the trigger is operated.

12. The device of claim 10, wherein said plunger control rod is rotatable to disengage said ratchet teeth and pawl and to thereby allow said plunger rod to be retracted distally by manual force applied to said plunger knob.

13. A method of injecting a therapeutic agent into a tissue site comprising manipulating a device as in any one of claims 1-12 to position the injection needle at said site while simultaneously visually observing the position of the needle on a patient external monitor associated with a video-assisted thorascopic system (VATS), advancing the needle into the tissue site and operating the device so as to expel a predetermined volume of the therapeutic agent through the needle and into the tissue site.

14. The method of claim 13, wherein the tissue is myocardium, and wherein the therapeutic agent includes transplanted cells.

15. The method of claim 13, further comprising positioning a stabilizing probe adjacent the tissue site.
